(19) United States
(12) Patent Application Publication
(10) Pub. No.: US 2006/0094974 A1
(43) Pub. Date: May 4, 2006

(54) Systems and methods for detecting brain waves

(76) Inventor: Robert C. Cain, St. Catharines (CA)

Correspondence Address: Joseph M. Sauer, Esq.;Jones Day, North Point, 901 Lakeside Avenue, Cleveland, OH, 44114, US

(21) Appl. No.: 11/264,215

(22) Filed: Nov. 1, 2005

Related U.S. Application Data
(60) Provisional application No. 60/624,316, filed on Nov. 2, 2004.

(51) Int. Cl. A61B 5/04

(52) U.S. Cl. 600/544; 600/545; 128/903

(57) ABSTRACT

A system for monitoring brain waves includes a detection electrode that is detects brain waves and is located on the part of the ear that is in or above the ear canal. The detection electrode also generates a brain wave data signal. A reference electrode is included in the system, and operates to detect a reference signal and to generate a reference data signal. A monitor is also included and receives the brain wave data signal and the reference data signal. The detection electrode and reference electrode form an electrode pair, and the monitor processes the brain wave data signal and data reference signal to generate neurofeedback.

### CROSS-REFERENCE TO RELATED APPLICATION

This application claims the benefit of priority from U.S. Provisional Application No. 60/624,316, filed on Nov. 2, 2004. This prior application, including the entire written description and drawing figures, is hereby incorporated into the present application by reference.

### FIELD

The technology described in this patent document relates generally to the field of brain wave detection and monitoring devices. More particularly, it relates to monitoring brain waves from the ear.

### BACKGROUND

Brain waves or electroencephalographic (EEG) signals can be monitored in order to detect and diagnose numerous medical conditions. Brain wave detection and monitoring can also be used to detect what areas of the brain are functioning, and to some extent, detect what a person is thinking. This information can be utilized in many useful applications.

Brain waves or EEG signals have been measured from various points on the scalp of a subject. Some devices use electrodes that are embedded within the patients scalp, while others use electrodes that are attached to the surface of the subjects skin. Embedding the electrode entails a surgically invasive procedure and attaching electrodes to the scalp can be aesthetically unpleasant. Furthermore, the electrodes are generally wired to a monitor that is located elsewhere on the body, and these wires and monitors are also aesthetically unpleasant, restrict movement, and the wires may become entangled.

### BRIEF DESCRIPTION OF THE DRAWINGS

FIG. 1 is a diagram of an example system for measuring brain waves from the ear.

FIG. 2 is a perspective view of a human ear.

FIG. 3a is a diagram of an example system for measuring brain waves from the triangular fossa.

FIG. 3b is a diagram of an example system for measuring brain waves from the cymba conchae.

FIG. 3c is a diagram of an example system for measuring brain waves from area straddling the triangular fossa and cymba conchae.

FIG. 4 is a diagram of a second example system for measuring brain waves from the ear.

FIG. 5 is a diagram of a third example system for measuring brain waves from the ear.

FIG. 6 is a partial cross-section of an example system for measuring brain waves from the ear canal.

FIG. 7 is a diagram of an example system for transmitting brain wave data wirelessly to a monitor.

FIG. 8 is a diagram of a second example system for transmitting brain wave data wirelessly to a monitor.

FIG. 9 is a diagram of a third example system for transmitting brain wave data wirelessly to a monitor.

FIG. 10 is a diagram of an example brain wave monitoring and alerting device.

FIG. 11 is a diagram of an example system for measuring brain waves from the ear canal where the detection electrode and the monitor are combined in the same housing.

### DETAILED DESCRIPTION

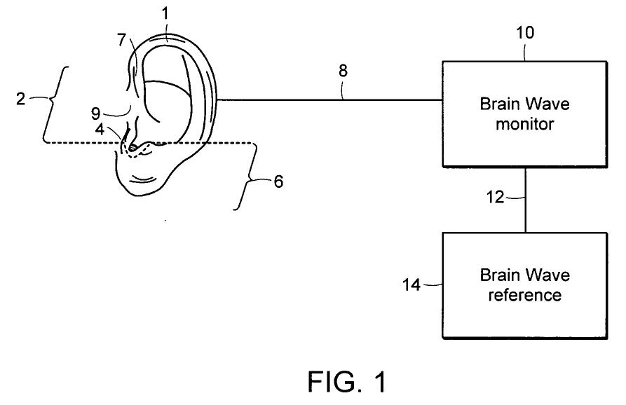

FIG. 1 is an example of a system for measuring brain waves from the ear 1. The ear 1 offers a relatively inconspicuous location, and has been found to be a site where brain wave activity is detectable. Certain areas of the ear 1 such as the area above the ear canal 2 and in the ear canal 4 have proven to be a better locus for detecting brain wave activity than the lower part of the ear 6. In particular, the area of the upper part of the ear 2 called the triangular fossa 7 and also the area called the cymba conchae 9 have been discovered to have especially high brain wave activity, especially near the skull. It is believed that the thinness of the skull at this location is the reason for the higher brain wave activity readings. Additionally, the triangular fossa 7 and cymba conchae 9 both form a type of natural basket for seating an electrode or monitoring device on the ear.

FIG. 1 shows a detected brain wave signal being communicated 8 to a brain wave monitor 10. The monitor 10 is also receiving a reference signal communication 12 from a brain wave reference source 14. The reference source 14 is typically an electrode placed in an area that displays little or no brain wave activity at all, or little or no brain wave activity of the same channel that is being measured. Examples of good locations for the reference electrode where there is little or no brain wave activity include the ear lobe, the outer part of the ear, the mastoids, and the chin. It is also possible to locate the reference source 14 in certain parts of the ear where brain wave activity is present but is not in the same channel of brain activity that the detection electrode is measuring. The reference source 14 serves as a ground for the monitor 10, enabling the monitor 10 to process the detection signal 8 into meaningful neurofeedback data by using the reference signal 12 as a zero point to compare with the detection signal 8.

The brain wave monitor 10 receives the brain wave signal 8 and the reference signal 12, and processes these signals together, using the reference signal 12 as a baseline. The monitor 10 may also amplify the signals 8, 10 to obtain better data. After processing, the monitor 10 outputs neurofeedback, which will allow users and observers to gain valuable data about the brain function of the user. This neurofeedback can be made available to the user/observer in a number of ways, some of which are discussed in detail below. The monitor 10 may be located, for example, behind the ear, in the ear, on a pair of glasses, hanging around the neck, on a belt, in a pocket, or wholly detached from the user's body.

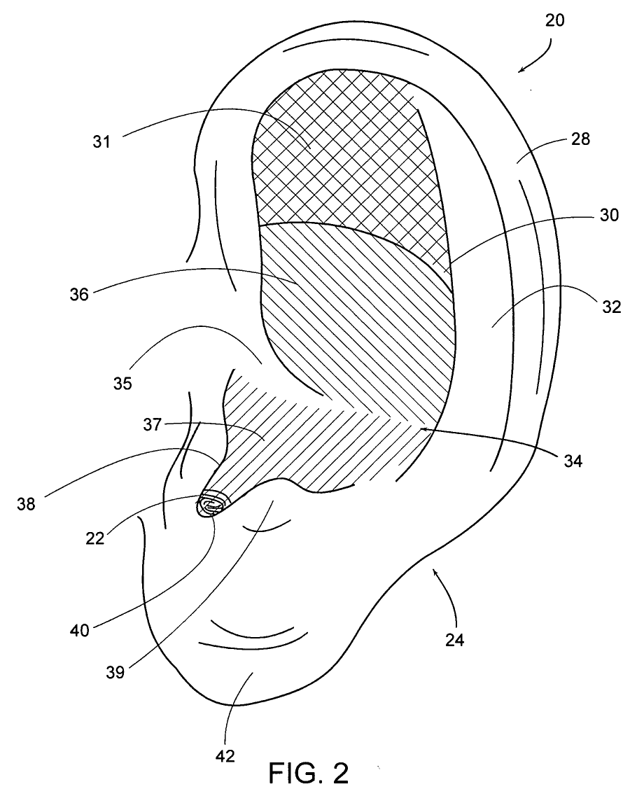

FIG. 2 shows the various anatomical parts of the human outer ear 20. The outer ear 20 includes the ear canal 22, also referred to as the auditory canal, and the pinna 24, also referred to as the auricle, which is the entire area surrounding the ear canal 22. The pinna 24 serves to collect vibrations from the air. The ear canal 22 conducts those vibrations to the ear drum of the middle ear.

The pinna 24 has various convex and concave formations. The outer edge of the pinna 24 has a prominent and curved rim called the helix 28. Running substantially parallel to the helix 28 is another curved prominence, the antihelix 30. The antihelix 30 widens and becomes less prominent at its upper terminus to form a triangular depression, known as the triangular fossa 31 (also referred to as the fossa triangularis). A narrow, curved depression located between the helix 28 and antihelix 30 is referred to as the fossa of the helix, or scapha 32. The antihelix 30 also curves around a semi-ovoid concavity called the concha 34. The concha 34 is divided by the commencement of the helix 35, (also known as the crus of the helix), into an upper part, the cymba conchae 36, and a lower part, the cavum concha 37. The concha 34 surrounds the ear canal 22 opening. Adjacent and partially opposed to the ear canal 22 opening is a pointed projection called the tragus 38. The antitragus 39 is located on the opposite side of the concha 34 from the tragus 38 and is also proximate to the ear canal 22 opening. A notch-like concavity, called the incisura intertragica 40, is positioned between the tragus 38 and antitragus 39. The ear lobe 42 is at the very bottom of the pinna 24, beneath the antitragus 39.

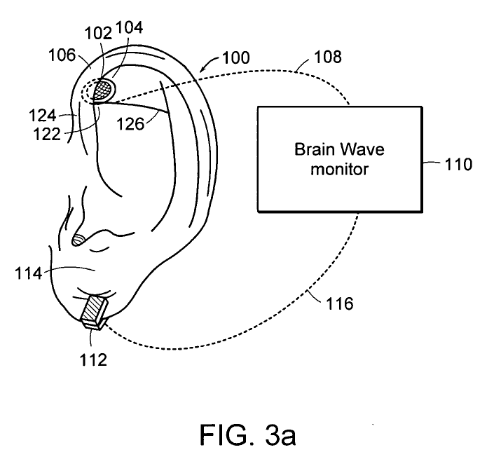

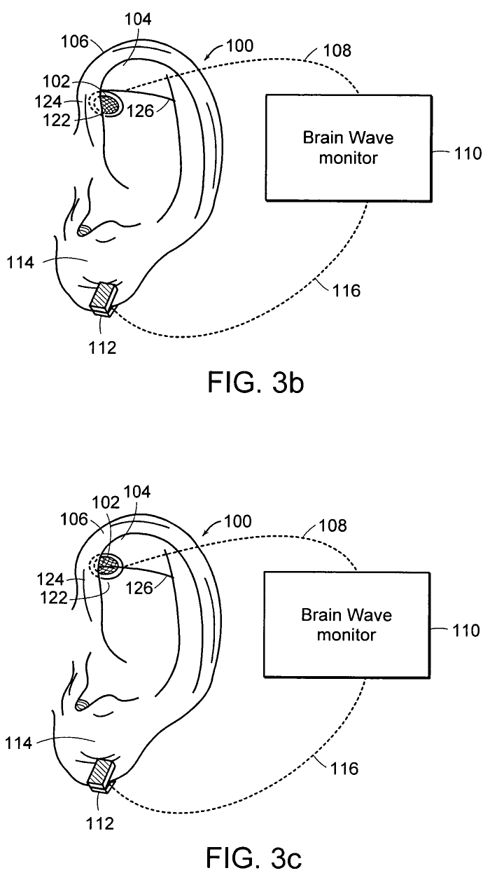

FIG. 3a is a diagram of an example brain wave monitoring system. A brain wave detection electrode 102 is positioned in the triangular fossa area 104 of the ear 100. In this example, the detection electrode 102 is partially situated underneath the helix 106 of the ear 100, in order to allow the electrode 102 to be positioned as close as possible to the skull. The detection electrode 102 is linked to a brain wave monitor 110, for example, by a wired connection 108.

FIG. 3a also depicts a reference electrode 112 that is located on the ear lobe 114 of the same ear 100 that the detection electrode 102 is located on. In this example, the reference electrode 112 is a clip, which allows it to be easily attached to the ear lobe 114. The reference electrode 112 is also linked to the brain wave monitor 110, for example by a wired connection 116. The monitor 110 performs processing and neurofeedback output functions.

FIG. 3b is a diagram of another example brain wave monitoring system. This system is the same as the system depicted in FIG. 3a, except the detection electrode 102 is located in the cymba conchae 122. In this example, the detection electrode 120 is partially concealed by the crus of the helix 124.

In another example, depicted in FIG. 3c, the detection electrode 102 may be located on both the cymba conchae 122 and the triangular fossa 126, straddling the lower ridge of the triangular fossa 104.

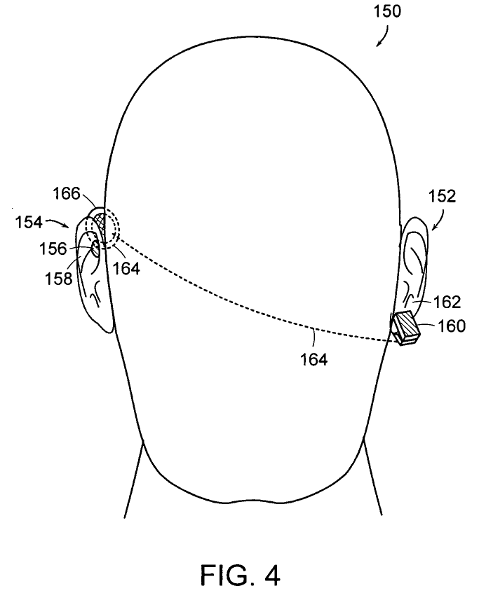

FIG. 4 shows a second example of a brain wave monitoring system positioned on a human head 150 having a left ear 152 and a right ear 154. A detection electrode 156 is located on the triangular fossa 158 of the right ear 154. A reference electrode 160 is positioned on the ear lobe 162 of the left ear 152. This example brain wave monitoring system is differentiated from the example shown in FIG. 3 partly by the fact that the reference electrode 160 is located on the opposite ear from the detection electrode 156. Locating the reference electrode 160 on the opposite side of the user's head may provide a better site for obtaining a reference signal that is more isolated from the brain wave activity channel that is being detected, and could increase the accuracy and usefulness of the neurofeedback.

Both the detection electrode 156 and the reference electrode 160 are linked, in this example, by a wire 164 to the monitor 166, which is located in a separate housing behind the ear. The monitor may be secured to the back of the ear, for example, by a clip, a hook, or an adhesive. The housing of the monitor 166 may also be shaped so that it will be held in place by frictional and gravitational forces alone. The monitor is depicted as being located behind the right ear 154, but in another example may be located on the left ear 152, or situated on the pinna of either ear 152, 154.

In another example, there is a brain wave monitoring system like the one shown in FIG. 4, except that it has a second pair of detection and reference electrodes. The second pair of electrodes is positioned on opposite ears from the pair of electrodes shown in FIG. 4. That is, the second detection electrode is positioned on the left ear 152 and the second reference electrode is positioned on the right ear 154. The second electrode pair, in one example, may be linked by a wired connection to the same monitor 166 shown in FIG. 4, or in another example, the second pair of electrodes may be linked by a wired connection to a second monitor, which may be located behind the left ear 152, opposite the monitor 166 of FIG. 4.

In another example, the system depicted in FIG. 4 is combined with other electrodes located at other positions on the ear or on the scalp of the user, and each electrode is linked to the monitor 166.

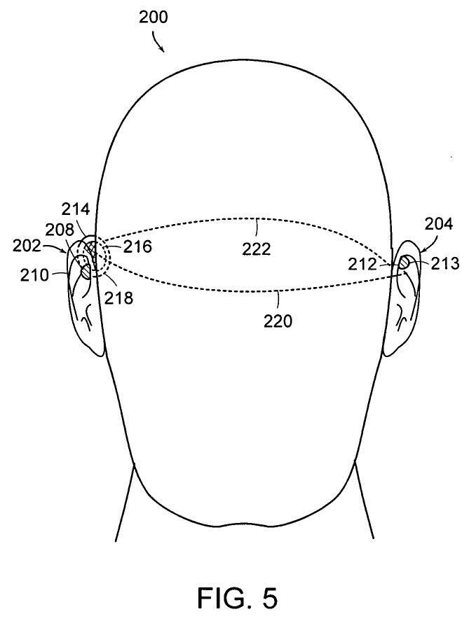

Referring now to FIG. 5, it shows a third example of a brain wave monitoring system positioned on a human head 200 having a right ear 202 and a left ear 204. A first combined electrode 208 is positioned on the triangular fossa 210 of the right ear 202. A second combined electrode 212 is positioned on the triangular fossa 213 of the left ear 204. These combined electrodes 208, 212, operate as both detection and reference sources and are processed relative to the opposite ear counterpart. That is, the detection source in the first electrode 208 is processed with the reference source in the second electrode 212, and the detection source in the second electrode is processed with the reference source in the first electrode. The first and second combined electrodes 208, 212 communicate with the monitor 214. The first combined electrode 208 sends a channel x detection signal 216 and a channel y reference signal 218 to the monitor 214. The second combined electrode 212 sends a channel y detection signal 220 and a channel x reference signal 222 to the monitor 214. The monitor 214 processes the channel x signals together, and the channel y signals together to output two sets of neurofeedback. The monitor 214 may also perform further operations on the data to average the two sets of neurofeedback. This dual arrangement allows collection of better neurofeedback than a single electrode pair would allow.

In another example, the system depicted in FIG. 5 is combined with other electrodes located at other positions on the ear or on the scalp of the user, and each electrode is linked to the monitor 214.

In another example, a second monitor is provided and channel x signals 216, 222 are transmitted to one monitor, while channel y signals 218, 220 are transmitted to the other monitor. Each monitor processes the signals separately.

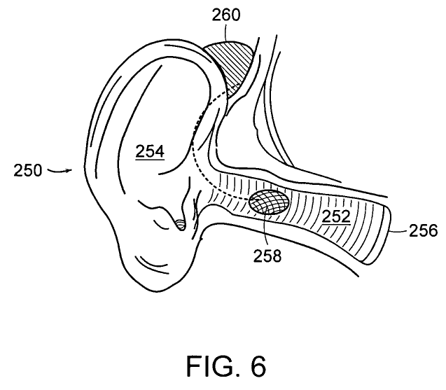

FIG. 6 shows a system for monitoring brain waves from the ear canal 252. A cross-section of the pinna 250 and ear canal 252 are presented. The ear canal 252 is an ovoid cylindrical passage that extends from the interior of the concha 254 to the ear drum 256. When measured from the surface of the concha 254 to the ear drum 256, the ear canal 252 is about an inch long. The ear canal 252 forms a gradual “S-shaped” curve and is directed, at first, inward, forward, and slightly upward, this is called the pars externa. The ear canal 252 then passes inward and backward, known as the pars media. The final part, known as the pars interna, passes inward, forward, and slightly downward.

FIG. 6 shows a detection electrode 258 located in the ear canal for detecting brain waves. This location provides at least the benefit of hiding the electrode from view, thereby increasing the aesthetic appearance of the monitoring system. A reference electrode is also included in the system and may be located in an appropriate place as discussed above. The detection electrode 258 and reference electrode are connected to the monitor 260 that processes the signal. If a wired connection is used, a clear, translucent wire may be used to link the detection electrode 258 to the monitor 260.

In another example, another detection electrode may be placed in the opposite ear and linked to either the monitor of FIG. 6 or to a second monitor. In yet another example, the monitor 260 and detection electrode 258 may be combined in a single earpiece wherein the electrode portion is located within the ear canal 252, and all or a portion of the monitor 260 is located outside the ear canal 252 and within the pinna 250.

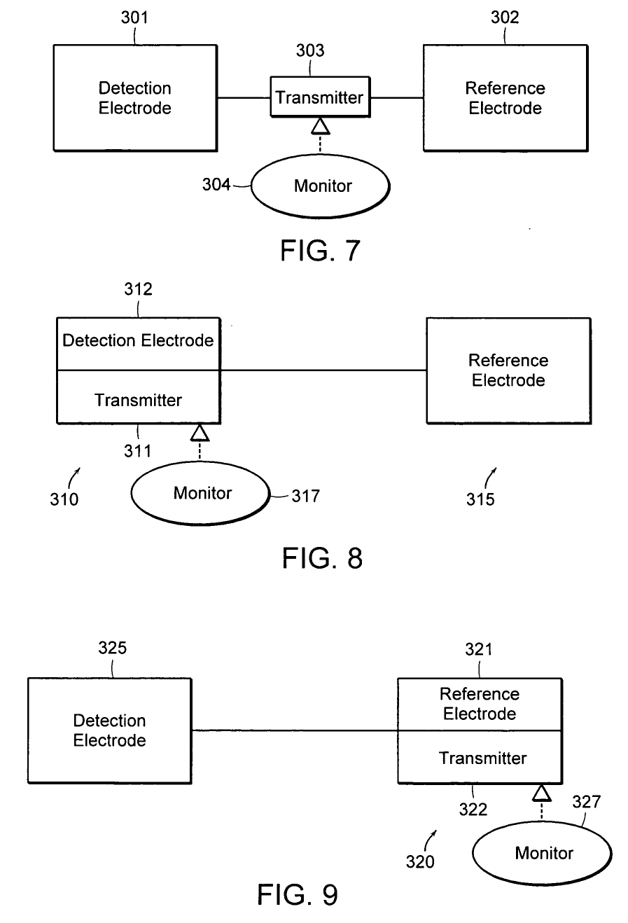

FIG. 7 is a diagram of an example wireless brain wave detection system. Wirelessly transmitting brain wave data will enhance the aesthetic appearance of the system, and prevent the problem of wires becoming entangled. An example of the wireless circuitry that can be used for this function is disclosed in U.S. patent application Ser. No. 11/100732 titled, “Binaural Hearing Instrument Systems and Methods,” which is hereby incorporated by reference in its entirety. The system includes a detection electrode 301, and a reference electrode 302. The data gathered by the detection 301 and reference electrodes 302 is transmitted to a wireless transmitter 303 that has an antenna and communications circuitry capable transmitting, or may first amplify then transmit the combined reference and detection electrode signal to a monitor 304. The detection electrode 301 may be placed on various locations on a patient where brain wave activity is detectable, including, for example, the triangular fossa, cymba conchae, and scalp. The reference electrode 302 may be placed in various places where there is little or no brain wave activity of the channel that is being measured by the paired electrode. Furthermore, multiple detection and reference electrodes may be added to the system and linked to the wireless transmitter 303 that is wirelessly linked to the monitor 304. The monitor 304 could also be placed in many different locations on the user or the user's clothing, or could even be located apart from the user, however, the monitor 304 should be within the wireless communication range of the transmitter 303 to properly receive data.

FIG. 8 shows a diagram of a wireless brain wave detection system that is similar to the example shown in FIG. 7. In FIG. 8, however, there is a combination detection electrode and transmitter 310. Both a detection electrode 312 and a transmitter 311 are included in the same housing. Communications circuitry and an antenna are also housed in the combination detection electrode and transmitter 310. A reference electrode 315 is also part of the system, and is linked to provide reference signal data to the combination detection electrode and transmitter 310. The reference and detection signals are combined and the transmitter 311 wirelessly transmits a combined data signal to a monitor 317. The combined signal may also be amplified before being wirelessly transmitted. Other reference electrodes and detection electrodes may also be added to the system and linked to the transmitter 311 and monitor 317.

FIG. 9 shows a diagram of a wireless brain wave detection system that is similar to the example shown in FIG. 8. However, in FIG. 9 there is a combination reference electrode and transmitter 320, rather than a detection electrode and transmitter 310. Both the reference electrode 321 and the transmitter 322 are included in the same housing, along with communications circuitry and an antenna. A detection electrode 325 is also part of the system, and is linked to the combination detection electrode and monitor 320 for transmitting detection signal data to it. The reference and detection signals are combined and the transmitter 322 wirelessly transmits a combined data signal to a monitor 327. The combined signal may also be amplified before being wirelessly transmitted. Other reference electrodes and detection electrodes may also be added to the system and linked to the transmitter 322 and monitor 327.

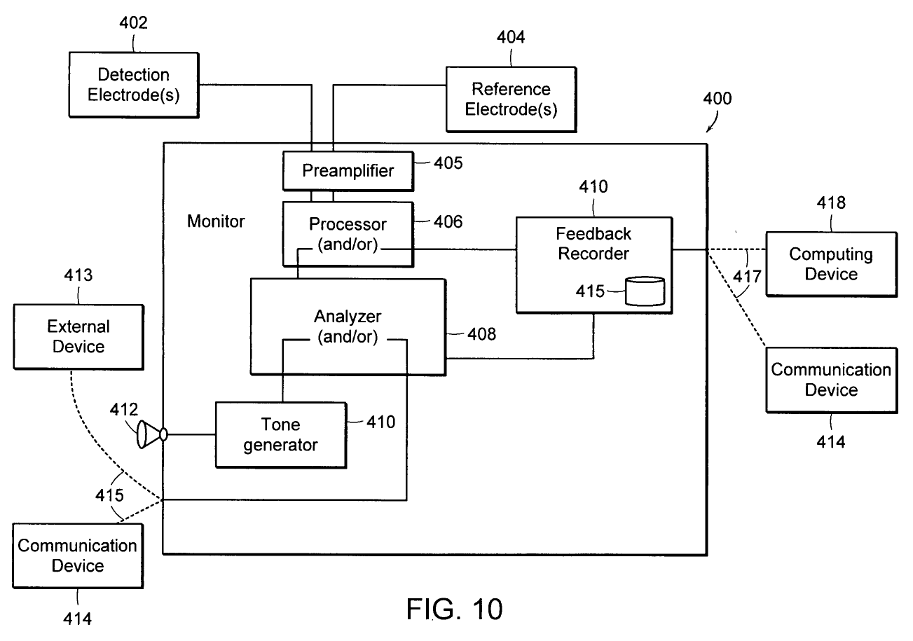

FIG. 10 shows an example of a system for alerting a user or a third party about dangerous brain wave activity. It also has recording and communication functions. The system includes a monitor 400 that is linked to one or more detection electrodes 402 and one or more reference electrodes 404. The monitor 400 houses a preamplifer 405 that is linked to the detection and reference electrodes and amplifies the signal that it receives. The amplified signal is transferred to a processor 406 which converts the brain wave data and reference signal data into neurofeedback data. One possible type of processor that may be used is Gennum Corporation's part number GC5055, also the processor could be the processor disclosed in U.S. patent application Ser. No. 11/100732, titled “Binaural Hearing Instrument Systems and Methods.” The neurofeedback data is then analyzed by the analyzer 408 to determine whether the neurofeedback represents a significant neural event. A significant neural event may be anything that a user or observer wishes to be notified of or have a record of. This allows for many useful applications, some of which are described below. If a significant neural event is detected, then the analyzer 408 triggers a warning mechanism; an example warning mechanism may be an audible tone and/or a signal to a communication device. The audible tone warning is generated by a tone generator 410 and emitted from a speaker 412. This would serve to alert the user, as well others in the vicinity if the tone is set loud enough for others to hear. Alternatively, or in conjunction with the alert tone, the analyzer 408 can transmit the neurofeedback data or an alert signal to a communication device 414 by a wired or wireless connection 415. The communication device 414, for example, could be a cellular phone, a wireless telephone connected to a land line, or a mobile network device. A signal 415 could also be sent to an external device 413. Alternatively, or in conjunction with the above features, the analyzer 408 could send neurofeedback data of only the significant neural event to the feedback recorder 416 described below.

As an alternative to or in conjunction with sending neurofeedback to the analyzer 408, the processor 406 may instead send neurofeedback data directly to a feedback recorder 416. The feedback recorder may record all neurofeedback data that is generated by the processor 406 and store it in memory 415, or if neurofeedback data is being sent directly to the analyzer, the feedback recorder 416 could accept neurofeedback data from the analyzer that represents a significant neural event and store only that data. The recording could be accessed by a wired or wireless connection 417 from a computing device 418 or a communication device 414. A computing device 418 may be, for example, a desktop computer, a personal data assistant, or a laptop. Accordingly, the neurofeedback could be transferred from the feedback recorder 416 at the user's convenience.

A system such as the one shown in FIG. 10 and described above, could be very useful as an epileptic early warning device. Epilepsy affects approximately 50 million people world-wide and 10-15% of people who suffer from epilepsy are untreatable. Epileptics have significantly reduced freedom in their lives as a result of never knowing when a seizure will occur and may not be able to drive a vehicle or operate machinery. Epileptics may also be restricted from participating in such things as swimming or having a bath for fear of drowning while having a seizure. Also, epileptics may fear social situations or public places, because of the embarrassment that having a seizure in public could cause.

An epileptic seizure is foreshadowed by a characteristic explosion of brain wave activity a few seconds before the seizure takes place. Normal waking consciousness results in slow wave 30-40 Hz, low voltage 20-30 μV eeg potentials. Seizures result in higher frequency and higher voltage 1000-2000 μV eeg potentials. The example system shown in FIG. 10 and described above could generate a warning signal to alert the user or a third party to the start of the seizure when the high frequency, high voltage brain waves are detected. The example FIG. 10 system can be applied to warn of oncoming seizures by programming or hardwiring the analyzer to recognize the characteristic brain wave activity of a seizure as the significant neural event. In turn, the significant neural event will trigger the warning mechanism, which will be either an audible alert generated by the tone generator 410 and speaker 412 or an alert signal that is transmitted to the communication device 414.

In the event the tone generator 410 is activated, the warning tone emitted from the speaker 412 would alert the user, and if the volume is turned up, persons in the vicinity of the user as well. This would allow the user a few seconds or even minutes to get out of a dangerous situation, such as a swimming pool or a moving vehicle. It could also allow the user to request help from those around him, or to get to a private place where they will not be so embarrassed to experience the seizure.

In another example, the tone generator 410, when triggered, emits vocal instructions that are loud enough to be heard by persons in the user's vicinity, and that state, for example, that the user is experiencing a seizure and requests help from those person in the surrounding area. This may be particularly useful for users who have especially serious epileptic seizures.

In another example, in the event the signal 415 to the communication device 414 is triggered, it may cause the communication device, for example, to call 911, or some other telephone number, or it may send an alert signal to a central monitoring site. It could also send an e-mail or other type of electronic communication over the internet. The signal could also cause certain information to be communicated to the contacted party as a request for help, such as an explanation of the emergency nature of the communication, and an identification of the user, etc.

In the event that the signal 415 to the external device 413 is triggered, it may cause the device 415 to shut down to minimize the chances of injury resulting from the seizure. For example, many automobile accidents are caused by epileptic seizures, and the damage from such a seizure could be minimized by signaling the vehicle to come to a controlled stop. Other types of vehicles and equipment could also be signaled to shut down when an oncoming seizure is detected.

In another example system, a global positioning system indicator is located on or near the user, and is linked by a wired or wireless connection to the monitor 400. The user's location is then communicated as part of the signal 415 to the communication device 414, perhaps as part of a request for help.

Another application for the example device of FIG. 10 would be in diagnosing epilepsy. A seizure can be reliably diagnosed to be epileptic if the brain waves during the seizure can be observed. Often it is difficult to get an eeg record of a seizure while it is happening, because the patient must be in a doctor's office or hospital and wired to an eeg monitor at the time the seizure occurs. Seizures are unpredictable and thus this is an unreliable and time consuming method of getting an eeg recording of a seizure. During this monitoring period the patient may also be induced to have a seizure by taking the patient off of their medication or by sleep deprivation or some other uncomfortable stimulus. With the example device of FIG. 10, however, the patient could go about their day outside the doctor's supervision, and a record of the seizure could be obtained whenever it naturally occurs. The brain wave data would be stored in the feedback recorder 416, and could be accessed by linking the monitor 400 to a computing device 418 or a communication device 414 by a wired or wireless connection 417.

Applications for other medical conditions that involve brain wave functions are also possible with the system of FIG. 10. For example, applications for disorders such as schizophrenia, narcolepsy, migraine headaches, and sleep disorders could all have similar functions as the epilepsy example, i.e. the warning tone function, the brain wave recording function, and the communication function.

The diagnosis and treatment of sleep apnea may be another especially useful application of the example device of FIG. 10. The monitor 400 could monitor and record brain waves that are indicative of sleep apnea, enabling physicians to diagnose the disorder while allowing the patient greater comfort and mobility. Furthermore, the warning tone function could be used to alert severe sufferers of sleep apnea when they are not breathing. This would be done by programming or hardwiring the analyzer 408 to recognize brain waves that indicate the user has stopped breathing, and set this as the significant neural event. This would then trigger the tone generator 410 to emit an alert tone through the speaker 412.

Applications for other uses that have definable brain wave patterns are also possible with the system of FIG. 10. For example, sleep and dream research, altered state research, stress management, relaxation training, and peak performance training. All these areas could benefit from at least the recording or communication functions of the example device of FIG. 10. Furthermore, the aesthetically pleasing and mobile nature of the device of FIG. 10 would encourage persons to participate in such research. Other applications, such as detecting brain waves for hands-free device control could also be made more aesthetically pleasant and mobile with the example device.

It should be understood that whenever an element is discussed as being located on a left or a right ear, it may also be located on the opposite ear. It should also be understood that any of the examples disclosed may be combined with a further array of electrodes. For example, a third electrode known as a “common” or a “right leg driver” would be beneficially combined with all of the above examples. Furthermore, while various features of the claimed invention are presented above, it should be understood that the features may be used singly or in any combination thereof. Therefore, the claimed invention is not to be limited to only the specific examples depicted herein.

Further, it should be understood that variations and modifications may occur to those skilled in the art to which the claimed invention pertains. The disclosure may enable those skilled in the art to make and use embodiments having alternative elements that likewise correspond to the elements of the invention recited in the claims. The scope of the present invention is accordingly defined as set forth in the appended claims.

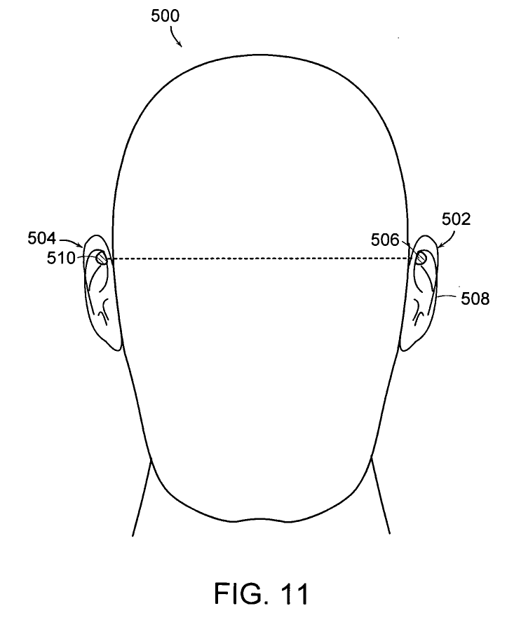

As an example of an alternative embodiment, FIG. 11 shows another example of a brain wave detecting system. A human head 500 is depicted having a left ear 502 and a right ear 504, and a combined detection electrode and monitor 506 is housed in the pinna 508 of the left ear 502. A detection electrode 510 is also located in the right ear 504 as well. The detection electrode 310 is linked to the monitor portion of the combined electrode and monitor 506. Reference electrodes that correspond to the detection electrode 510 and the detecting electrode portion of the combined electrode and monitor 506 maybe located in the appropriate places that have been discussed in this specification.

In another example, the combined electrode and monitor 506 of FIG. 11 also includes a reference electrode that corresponds to the detection electrode 510 on the opposite ear 504. The detection electrode 510 may also include a reference electrode so that it is a combined electrode like that depicted in FIG. 5. In yet another example, the ears 504, 502 may both contain a combined detection electrode, reference electrode, and monitor. In this case, each monitor portion would process a separate set of signals.

It is claimed:

1. A system for monitoring brain waves comprising:
a detection electrode operable to detect brain waves; the detection electrode being operable to communicate a brain wave data signal to a brain wave monitor;
a reference electrode operable to detect a reference signal; the reference electrode being operable to communicate a reference data signal to the brain wave monitor;
the brain wave monitor including communications circuitry for communicating wirelessly.

2. The system of claim 1 wherein the brain wave monitor is situated in a housing, and one of the detection or reference electrodes is also situated in the same housing.

3. The system of claim 1 further comprising: a plurality of said detection and reference electrodes, so that each reference electrode is paired with a detection electrode to form an array of pairs of electrodes.

4. The system of claim 3 wherein at least two of said pairs of electrodes include one electrode that is situated in the same housing as the brain wave monitor.

5. The system of claim 1 wherein at least one detection electrode is located in a triangular fossa area of an ear.

6. The system of claim 1 wherein at least one detection electrode is located in a cymba conchae area of an ear.

7. The system of claim 1 wherein at least one detection electrode is located in both a triangular fossa and cymba conchae area of an ear and straddling a lower ridge of the triangular fossa.

8. The system of claim 1 wherein at least one detection electrode is located in the area of an ear that is covered by a helix or covered by a crus of the helix.

9. A system for monitoring brain waves comprising:
a detection electrode operable to detect brain waves and located on the ear of a user; the detection electrode being operable to communicate a brain wave data signal to a brain wave monitor;
a reference electrode operable to detect a reference signal; the reference electrode being operable to communicate a reference data signal to the brain wave monitor;
wherein the monitor is operable to process the brain wave data signal and reference electrode signal into neurofeedback, and to trigger a warning mechanism when a significant neural event occurs.

10. The system of claim 9 wherein the warning mechanism is a tone generator that generates an audible warning signal connected to a speaker.

11. The system of claim 10 wherein the audible signal is a request for help.

12. The system of claim 10 wherein the warning mechanism is an alert signal sent by a wired or wireless link to a communication device.

13. The system of claim 12 wherein the alert signal causes the communication device to dial an emergency telephone number.

14. The system of claim 13 wherein the alert signal further causes a request for help to be sent to the dialed emergency number.

15. The system of claim 12 wherein the alert signal causes the communication device to send an electronic message.

16. The system of claim 9 wherein the warning mechanism is a signal sent by a wired or wireless link to an external device.

17. The system of claim 16 wherein the signal causes the external device to shut down.

18. The system of claim 17 wherein the external device is an automobile.

19. The system of claim 9 further comprising a feedback recorder, wherein the neurofeedback is transmitted to a feedback recorder for storage in memory.

20. The system of claim 19 wherein only neurofeedback representing one or more of the significant neural events is transmitted to the feedback recorder for storage in memory.

21. The system of claim 9 wherein neurofeedback is transmitted by a wired or wireless link to a communication device.

22. The system of claim 9 wherein the significant neural event is the characteristic neurofeedback preceding an epileptic seizure.

23. A system for monitoring brain waves comprising:
a detection electrode operable to detect brain waves, and located at any part of an ear that is in or above an ear canal of said ear, and operable to generate a brain wave data signal;
a reference electrode operable to detect a reference signal, and operable to generate a reference data signal;
the detection electrode and reference electrode forming an electrode pair
a monitor operable to receive the brain wave data signal and the reference data signal;
wherein the monitor is operable to process the brain wave data signal and data reference signal to generate neurofeedback.

24. The system of claim 23 wherein the detection electrode is located at the triangular fossa of an ear.

25. The system of claim 23 wherein the detection electrode is located on the area of a triangular fossa that is closest to a front part of a user's head and partially enclosed by a crus of the helix.

26. The system of claim 23 wherein the detection electrode is located at a cymba conchae of an ear.

27. The system of claim 23 wherein the detection electrode is located in the ear canal.

28. The system of claim 23 wherein the monitor further comprises a neurofeedback recorder that is operable to store in memory the generated neurofeedback.

29. The system of claim 28 wherein the memory containing the stored neurofeedback is accessible by one of a computing device or communication device via a wired or wireless link.

30. The system of claim 23 wherein at least one of the reference electrode or the detection electrode is included in a housing with the monitor.

31. The system of claim 23 further comprising: a plurality of said detection and reference electrodes, so that each reference electrode is paired with a detection electrode to form an array of pairs of electrodes.

32. The system of claim 23 wherein at least two of said pairs of electrodes include one electrode that is situated in the same housing as the brain wave monitor.

33. The system of claim 23 wherein the reference electrode and detection electrode are located on opposite sides of a user's head.

34. The system of claim 23 further comprising: a plurality of said detection and reference electrodes, so that each reference electrode is paired with a detection electrode to form an array of pairs of electrodes.

35. The system of claim 34 wherein within each pair of reference electrodes and detection electrodes, the reference electrode and detection electrode are located on opposite sides of a user's head.

36. The system of claim 35 wherein at least one reference electrode is contained in the same housing as at least one detection electrode, and the at least one reference electrode and at least one detection electrode are not in the same pair.

37. A method for detecting brain waves comprising the steps of:
detecting brain waves from a location at any part of an ear that is in or above an ear canal of said ear, and generating a brain wave data signal;
detecting a reference signal and generating a reference data signal;
transmitting the reference data signal and the brain wave data signal;
receiving the reference data signal and the brain wave data signal;
processing the reference data signal and the brain wave data signal to generate neurofeedback.

38. The method of claim 37 wherein the step of detecting brain waves is done from the triangular fossa.

39. The method of claim 37 wherein the step of detecting brain waves is done from the cymba conchae.

40. The method of claim 37 wherein the step of detecting brain waves is done from the area concealed by a helix and a crus of the helix.

41. The method of claim 37 further comprising the step of analyzing the neurofeedback to determine if a significant neural event is occurring.

42. The method of claim 41 further comprising the step of triggering a warning mechanism when a significant neural event occurs.

43. The method of claim 42 wherein the warning mechanism is an audible alert tone.

44. The method of claim 42 wherein the warning mechanism is transmitting an alert signal to a communication device.

45. The method of claim 42 wherein the warning mechanism is transmitting a signal to an external device.

46. The method of claim 45 wherein the signal causes the external device to shut down.

47. The method of claim 46 wherein the external device is an automobile.

48. The method of claim 41 wherein the significant neural event is an epileptic seizure.

49. The method of claim 37 further comprising the step of recording the neurofeedback.

50. The method of claim 41 further comprising the step of recording the neurofeedback that indicates the significant neural event is occuring.
